(19) United States
(12) Patent Application Publication
(10) Pub. No.: US 2006/0041022 A1
(43) Pub. Date: Feb. 23, 2006

(54) Treatment of amyotrophic lateral sclerosis with nimesulide

(76) Inventor: Giulio Maria Pasinetti, New York, NY (US)

Correspondence Address: BAKER & BOTTS, 30 ROCKEFELLER PLAZA, NEW YORK, NY, 10112, US

(21) Appl. No.: 11/122,351

(22) Filed: May 5, 2005

Related U.S. Application Data
(63) Continuation of application No. PCT/US03/08905, filed on Mar. 24, 2003.
(60) Provisional application No. 60/424,729, filed on Nov. 6, 2002.

(51) Int. Cl. A61K 31/18

(52) U.S. Cl. 514/602

(57) ABSTRACT

The present invention relates to methods for delaying the onset or progression of motor impairment associated with amyotrophic lateral sclerosis in a subject by administering to the subject a therapeutically effective amount of nimesulide. It further provides for a means of detecting and monitoring the progression of amyotrophic lateral sclerosis via a protein biomarker for the disease.

### GRANT SUPPORT

Not applicable.

### 1. INTRODUCTION

The present invention relates to methods for delaying the onset or progression of motor impairment associated with amyotrophic lateral sclerosis in a subject by administering to the subject a therapeutically effective amount of nimesulide. It further provides for a means of detecting and monitoring the progression of amyotrophic lateral sclerosis via a protein biomarker for the disease.

### 2. BACKGROUND OF THE INVENTION

Amyotrophic lateral sclerosis (ALS) is a fatal progressive motor neuron disorder that results in significant inflammation and neuron loss in the ventral horns of the spinal cord and, to a lesser extent, the brain. Approximately ten percent of ALS occurrences are genetic in origin (Mulder et al., 1986, Neurology 36(4):511-517; Siddique et al., 1989, Neurology 39(7):919-925; Sillevis Smitt et al., 1994, Biol Signals 3(4): 193-197) of which twenty percent are associated with mutations in the antioxidant copper, zinc superoxide dismutase-1 (SOD)1 gene (Rosen et al., 1993, Nature 362(6415):59-62).

A murine model of SOD1 mutation-associated ALS has been developed in which mice express the human SOD mutation glycine→alanine at residue 93 (SOD1). These “SOD1” mice exhibit a dominant gain of the “adverse property” of SOD, and develop motor neuron degeneration and dysfunction similar to that of human ALS (Gurney et al., 1994, Science 264(5166):1772-1775; Ripps et al., 1995, Proc Natl Acad Sci U.S.A. 92(3):689-693; Bruijn et al., 1997, Proc Natl Acad Sci U.S.A. 94(14):7606-7611). Features common to human ALS include astrocytosis, microgliosis, oxidative stress, increased levels of cyclooxygenase/prostaglandin, and, later in the disease process, profound motor neuron loss.

Increasing anecdotal evidence suggests that, based upon antioxidant and anti-inflammatory properties, non-steroidal antiinflammatory drugs (“NSAIDs”) may delay neuroinflammation in ALS and be useful in lessening the symptoms of motor dysfunction in this disease. U.S. Pat. No. 5,985,930 by Pasinetti and Aisen, and its international counterpart, International Patent Application No. PCT/US97/21484, Publication No. WO 98/22104, disclose and claim a method of preventing neuronal cell death in a patient suffering from ALS comprising administering, to the subject, an effective amount of nimesulide, a non-selective cyclooxygenase inhibiting NSAID with potent antioxidant properties. Nimesulide has been shown to be well tolerated in geriatric patients for periods greater than two years (Aisen et al., 2002, Neurology 58(7):1050-1054).

### 3. SUMMARY OF THE INVENTION

The present invention relates to methods for delaying the onset or progression of motor impairment associated with ALS in a subject comprising administering, to the subject, a therapeutically effective amount of nimesulide. It is based, at least in part, on the discovery that nimesulide was able to delay the onset of motor impairment in a murine model of ALS.

Accordingly, nimesulide may be used to prophylactically treat persons in the general population and more particularly persons believed to be at risk for developing ALS because of, for example, a positive family history for the disease and/or the presence of a genetic defect. In addition, nimesulide may be used to treat persons already diagnosed with ALS to delay the progression of existing motor impairment and/or to delay the onset of motor impairment in motor systems not yet detectably affected by the disease.

In another aspect of the invention, a biomarker for ALS motor impairment is identified which may be used as a means of diagnosing the disease and/or a monitoring its progression. It is based, at least in part, on the discovery that levels of a particular protein were found to increase in the spinal cord of SOD1 (ALS model) mice in a manner that correlated with the deterioration of motor skills.

### 4. BRIEF DESCRIPTION OF THE FIGURES

FIG. 1. Nimesulide delays the onset of motor impairment in SOD1 mutant mice as assessed by the rotarod. Graphical plot of accelerating rotarod test data demonstrating that although SOD1 mice showed an average onset of motor impairment on day 110 (red circles, n=6), SOD1/NMS mice (blue diamonds, n=6) supplemented with nimesulide showed a significant (P<0.05) 10-day delay (window) in the onset of motor impairment. A dashed line indicates the first date of onset of bilateral paralysis, such that (n) values begin to diminish beyond this day of testing due the lack of ability of some mice to perform the task. An arrow indicates the date of death of last ALS animal. Data prior to day 92 were unequivocal.

FIG. 2. Nimesulide delays the onset of motor impairment in SOD1 mutant mice as assessed by grid walking. Graphical plot of grid walking test data showing that SOD1 mice treated with nimesulide (blue diamonds, n=6) maintained motor ability for 10 days beyond SOD1 mice that had been fed a control diet (red circles, n=6; statistically significant on days 113, 115, 122 and 124 (window); P<0.05). A dashed line indicates first date of onset of bilateral paralysis, such that (n) values begin to diminish beyond this day of testing due lack of ability of some mice to perform the task. An arrow indicates the date of death of the last ALS animal. Data prior to day 92 were unequivocal.

FIG. 3A-B. Elevated expression of a 4.8 kDa positively-charged protein species in the spinal cord of SOD1 mice is modulated by NMS treatment. (A) SELDI retention map of negatively charged proteins in mouse spinal cord samples. Peaks represent individual proteins and the area under each peak represents the signal intensity. Reference molecular sizes are as indicated across the bottom of the panel. Red arrows indicate the 4.8 kDa protein species which is up-regulated in SOD1 transgenics compared to wild-type littermates, and is modulated by NMS treatment. (B) Graphical representation of 4.8 kDa protein content. Data are expressed as mean±SEM; *P<0.05, (n=4 per group).

FIG. 4A-B. Nimesulide and prostaglandin levels in nimesulide treated mice. (A) is a bar graph showing the nimesulide content in brain (solid bars) and serum (open bars) in SOD1 mice with or without nimesulide treatment. (B) is a bar graph showing prostaglandin E2 levels in wild-type (“WT”; solid bars) or SOD1 (open bats) mice which were untreated or treated with nimesulide.

### 5. DETAILED DESCRIPTION OF THE INVENTION

The present invention provides for methods for delaying the onset or progression of motor impairment associated with ALS in a subject comprising administering, to the subject, an effective amount of nimesulide.

“Nimesulide” is a term used to refer to a compound having a chemical structure as set forth in Formula I. 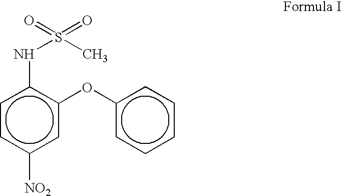
Using standard chemical nomenclature, this same compound is also referred to in the art as 4-nitro-2-phenoxymethanesulfonanilide.

According to the invention, nimesulide may be administered to any person in the general population as prophylaxis against the possibility that the person may in the future develop ALS. In preferred embodiments of the invention, nimesulide may be administered to a person suspected of being at risk for ALS, for example, by virtue of being in a family with a higher than normal incidence of ALS or due to a defined genetic proclivity, for example, as a result of a mutation in the SOD gene. Another category of subjects who may, in preferred embodiments of the invention, be prophylactically treated with nimesulide are persons who have experienced an environmental exposure believed to be associated with the development of ALS such as exposure to pesticides, herbicides, organic solvents, mercury, lead, manganese, or selenium, who smoke cigarettes or who have experienced trauma to the nervous system.

In addition, nimesulide may be administered to a subject in the early stages of ALS, preferally upon a determination that the diagnosis of ALS is probable. For purposes of definition, the period considered the “early stage” is the first year after the onset of symptoms.

In further embodiments, nimesulide may be administered to a subject in the later stages of ALS to delay the onset of symptoms in particular motor systems, for example, in order to delay impairment of vocalization and/or the respiratory musculature associated with dysfunction of cranial motor nerves. For purposes of definition, patients suffering from ALS for more than one year are in the later stages of the disease.

The amount of nimesulide administered may produce a local concentration, in the nervous system, of at least 10 (ten) nanomolar, more preferably at least 1 (one) micromolar. The amount of nimesulide administered may produce a serum concentration of at least 10⁻⁷ molar, preferably at least 10⁻⁶ molar, and more preferably at least 10 (ten) micromolar.

The amount of nimesulide administered per day may be 200 mg per day but preferably is less. The present invention provides, in specific, non-limiting embodiments, for daily dosages of up to 200 mg, between 100 and 200 mg (e.g. 100 mg), between 50 and 100 mg (e.g., 50 mg), or between 10 and 50 mg (e.g. 20 mg) (all ranges are inclusive of their limits). The daily dosage may be administered as a single dose or as divided doses.

In preferred embodiments of the invention nimesulide is administered orally but other routes of administration, including subcutaneous, inhalation, intravenous, intrathecal, rectal, or any other suitable route may be used. The formulation containing nimesulide may be varied using standard methods and compounds depending on the mode of administration.

The present invention may also be practiced by administering the foregoing daily dosages such that there are days where the subject “skips” treatment—for example, the daily dosage is administered every other day, or every third day, etc.

It is desirable to monitor a subject's liver function tests and stop or suspend treatment if abnormalities arise.

Nimesulide may be administered according to the invention for any desirable duration of time. The “treatment period” is preferably, but not by way of limitation, at least six months.

In another aspect of the invention, nervous system tissue or cerebrospinal fluid of a subject may be tested for the presence of an ALS biomarker. As one specific non-limiting embodiment, protein from cerebrospinal fluid may be applied to ProteinChip Arrays with varying surface chemical/biochemical properties and analyzed by surface enhanced laser desorption ionization time of flight mass spectrometry (see Section 6, below). The presence of a protein having a molecular weight of between about 3.5-6.5 kDa; preferably between about 4.5 and 6.0 kDa, which is negatively charged at pH 9 and which is absent or significantly decreased in a comparable sample from a healthy control subject correlates positively with the diagnosis of ALS. Further, in a series of samples obtained from the same subject over time, an increase in the amount of said protein positively correlates with the progression of motor impairment. The existence of such a biomarker facilitates the diagnosis of the disease and monitoring its progression.

In related embodiments the present invention provides for assay systems which may be used to identify agents for the treatment of disorders in motor function, including but not limited to the motor function impairment associated with ALS. In particular non-limiting examples, the level of a 4.5-6.0 kDa, preferably an approximately 4.8, 5.3 or 5.7 kDa protein, which is negatively charged at pH 9, in murine models of motor impairment may be used to determine whether a test agent, administered to such mice or to cultures of tissue obtained from such mice, has a beneficial effect on preventing, delaying, or decreasing motor impairment. For example, an SOD1 mouse may be administered a test agent for a period of time, and then the level of the 4.5-6.0, preferably 4.8, 5.3 or 5.7 kDa “biomarker” protein in the spinal cord and/or spinal fluid of the mouse may be determined and compared with control mice that either did not receive the test agent or were administered a different dose of test agent. The level of said protein may be monitored over different treatment intervals. The ability of the test agent to delay or inhibit the accumulation of the biomarker protein, particularly in a dose-dependent manner, may be indicative of a therapeutic benefit. The effect of the test agent on the biomarker may be correlated with the effect of the test agent on motor skill performance by the test animal(s).

### 6. EXAMPLE

### 6.1 MATERIALS AND METHODS

Mice Studies were performed on 8 week old female mice overexpressing mutated human superoxide dismutase (codon 93 glycine→alanine G93A (TgN[SOD1-G93A]1Gur(Gurney et al., 1994, Science 264(5166):1772-1775) Jackson Laboratory, Bar Harbor, Me.; “SDO1 mice”) and their wild type littermates. Mice were housed on a 12 hour day/light cycle and (beginning at 45 d of age) allowed ad libitum access to either nimesulide (NMS)-supplemented (19 g/10 kg) chow, delivering ˜1.5 mg/g per day to the animal, or, as a control, plain NIH-07 formula cold press chow processed into identical ½ inch pellets (Zeigler Bros Inc, Garners, Pa.). Genotyping was conducted at 21 days of age as described in Gurney et al., 1994, Science 264(5166):1772-1775.

Rotarod and Grid Walking Tests. To assess balance, coordination and muscle strength mice were tested on the accelerating rotarod (7650 Ugo Basile Biol. Res. App., Italy) and grid walking tests. For the rotarod test, mice were placed onto a grooved cylinder rotating at a predetermined speed that incrementally increased to a maximal rotation at 180 seconds; the time maintained on the rod by each mouse was recorded (180 max). For the grid walking test, animals were placed into the distal end of a walled chamber (15 cm W×60 cm L, 20 cm high walls and a wire mesh bottom) suspended 1 meter above the floor. The number of foot misses (entire paw and portion of limb pokes through the wire mesh) while crossing a distance of 60 cm was recorded. Beginning at 82 days of age, mice were tested on both tasks three times per week until SOD1 groups could no longer perform the tests, evaluators were blind to diet treatment at all stages of experimentation.

Health/Neurologic Status. To assess health status mice were weighed weekly and examined for changes in lacrimation/salivation, palpebral closure, ear twitch and pupillary responses, whisker orienting, postural and righting reflexes and Body Condition Score (BCS) (Ullman-Cullere 1999). Finally, a general pathological examination was conducted at time of sacrifice. NMS was found to have no effect on weight in either SOD1 of wild type animals. All health/neurologic tests were unremarkable, with the exception of the SOD1 groups which both, as expected, exhibited decreased BCS scores and impaired postural and righting reflexes following the onset of hind limb paralysis (˜122 d).

Pathology examination. To evaluate molecular changes during the period of therapeutic efficacy of NMS, a sub group of mice were sacrificed by cervical dislocation 95-105 d of age. Blood, brain and lumbar spinal cord samples were then rapidly harvested and prepared for analysis.

Nimesulide and Prostaglandin E₂. Nimesulide levels in the serum and rostral hemisphere of the brain were assessed by high performance liquid chromatography, and found to be in accord with our previous studies demonstrating that 10% of the serum levels of NMS crossed the blood brain barrier. Prostaglandin-E₂ expression in the left caudal hemisphere of the brain (including substantia nigra, and motor cortex) was measured by immuno-assay (Cayman Ann Arbor, Mich.), which was performed as follows. Briefly, pulverized brain tissue stored in liquid N₂ was homogenized in 0.1M phosphate-buffered saline (containing 1 mM EDTA and 10 μM indomethacin), mixed with an equal volume of ethanol, and centrifuged. The supernatant was diluted with 50 mM acetic buffer and purified through an affinity column (Cayman). The column was equilibrated with column buffer (0.1M phosphate-buffered saline, 7.7 mM NaN₃, 0.5 M NaCl₂) followed by UltraPure water, the supernatant was then eluted from the 4 ml column by adding the elution solution and allowing it to pass through the packing material. The eluate was then evaporated and re-dissolved in enzyme-linked immunoassay buffer, applied to a 96-well plate pre-coated with goat anti-mouse IgG and incubated with PGE₂ monoclonal antibody and (recovery tracer) for 18 hr at 4° C. After incubation with the PGE₂ monoclonal, the plate was rinsed fives times with washing buffer and developed using Ellman's reagent for 1 h at room temperature. The PGE₂ concentration was determined spectrophotometrically and calculated by plotting the standard % B/B0 (% sample or standard Bound/Maximum Bound) versus PGE₂ concentration (in pg/ml).

Biomarker Analysis. To assess the regulation of protein biomarkers in SOD1 mice during the onset of motor impairment, samples of lumbar spinal cord (protein extracts) were applied to ProteinChip Arrays with varying surface chemical/biochemical properties and analyzed by surface enhanced laser desorption ionization time of flight mass spectrometry (Ciphergen, Fremont Calif.). Then, using integrated protein mass profile software, gathered data was used to compare protein expression profiles of the various treatment groups.

Statistics. Statistical analysis was performed using the StatSoft software package (StatSoft). Students' t-test was used to test the significance between differences in mean values. For all analyses the null hypothesis was rejected at p<0.05.

### 6.2 RESULTS

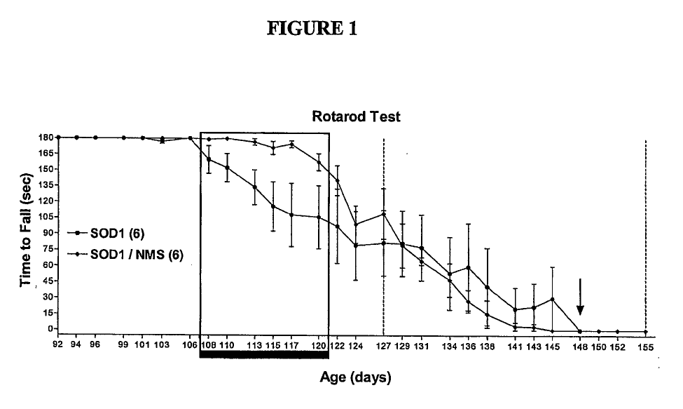

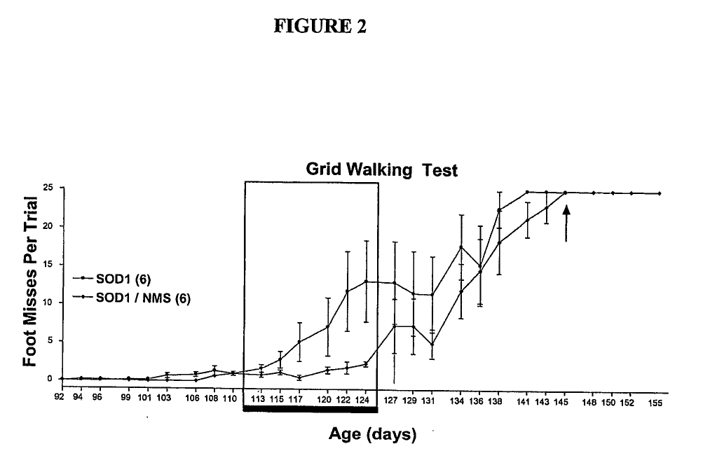

Motor Ability as a Function of the Therapeutic Efficacy of NMS. SOD1 mice fed a control diet exhibited a mean onset of motor impairment at 108 days of age in the rotarod (FIG. 1) and grid walking (FIG. 2) tests. In contrast, SOD1 mice fed a diet supplemented with NMS exhibited motor skill integrity until reaching 120 days of age in the rotarod test (FIG. 1) and 124 days of age in the grid walking test (FIG. 2). Thus, the NMS-treated SOD1 mice exhibited a significant delay in the onset of impairment relative to their untreated counterparts. Following this period of delay, performance of SOD1 mice treated with NMS deteriorated to levels similar to that of control SOD1 mice. Wild type groups displayed optimum performance scores of 180 seconds throughout the testing period.

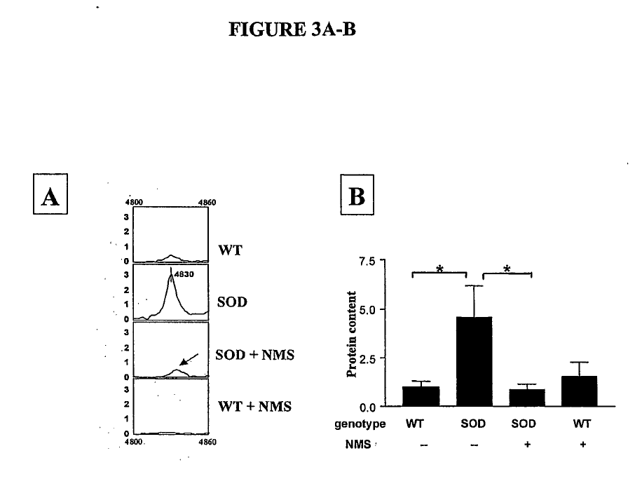

Biomarker and Prostaglandin Levels as a Function of the Therapeutic Efficacy of NMS. From lumbar spinal cord protein extracts collected immediately prior to the onset of motor impairment in SOD1 mice, and therapeutic delay of impairment in SOD1/NMS mice, a total of 19 proteins with altered regulation were identified, compared to wild type littermates. Of these proteins, a protein having a molecular weight of approximately 4.8 kDA protein (negatively charged at pH 9) was found to be significantly elevated in the spinal cord of SOD1 mice prior to the onset of motor impairment (˜90 d of age), and was regulated back to control levels in SOD1 mice that received NMS treatment (FIGS. 3A and B). Proteins having molecular weights of approximately 5.3 and 5.7 kDa were observed to increase with disease progression and decrease with nimesulide treatment.

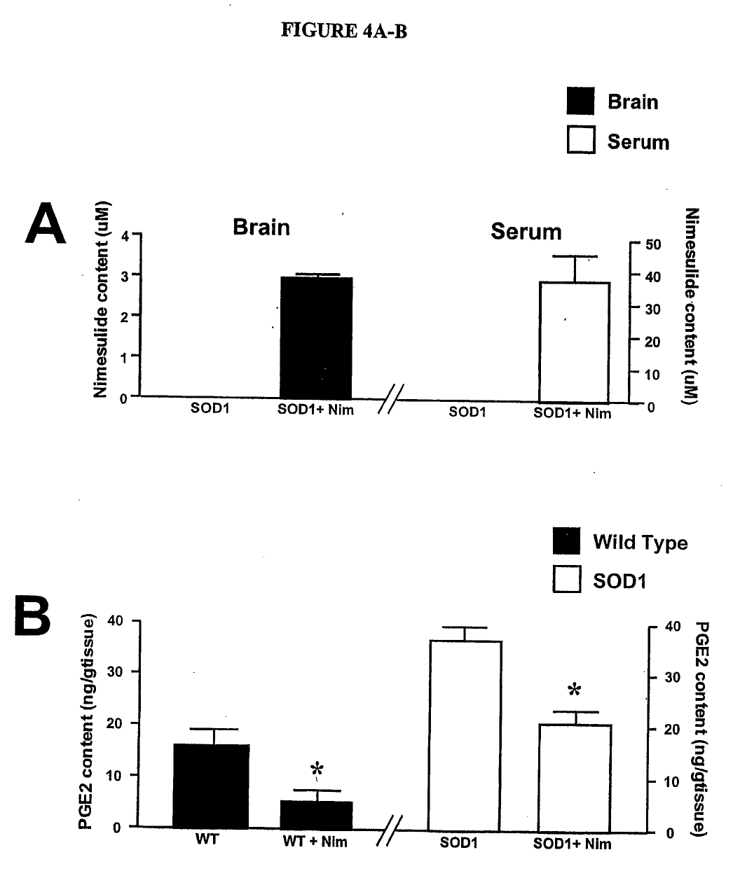

Prostaglandin levels. At the onset of bilateral hind-limb paralysis (time of sacrifice) we found that the nimesulide delivery to mice through the feeding (2-3 months treatment) reached approximately 30 μM concentration in serum (FIG. 4A). Assessment of nimesulide in the spinal cord revealed that approximately 10% of serum levels were detectable in brain parenchyma (cerebral cortex). Consistent with previous evidence, in parallel studies we also found that the absolute concentration of PG-E2 content in the cerebral cortex of SOD1 mice relative to WT group assessed in separate experiments revealed >2 fold elevation (P<0.01). Most importantly, we found that prophylactic treatment of SOD1 (or WT) mice with nimesulide in the diet coincided with decreased PG-E2 content in the spinal cord (FIG. 4B).

Weight as an index of ALS disease progression. Mice were weighed weekly beginning at 6 weeks of age. No detectable difference in weight was found between SOD1 mice fed normal diet and SOD1 mice fed nimesulide diet. Following the onset of motor dysfunction (day 112), a significant groups difference was detected (F=6.95, P=0.009), such that the weight of both SOD1 mice fed normal diet and SOD1 mice treated with nimesulide were significantly lower than that of WT control (p<0.01 for both groups). WT control littermate mice were found to maintain similar mean weights throughout the duration of testing, and exhibited a 10% increase by 109 days of age (when compared to week 6), and a 17% increase by 122 days of age.

Various publications, including patents, patent applications, and non-patent publications are cited herein which are hereby incorporated by reference in their entireties.

What is claimed is:

1. A method of delaying the onset of motor impairment associated with amyotrophic lateral sclerosis in a human subject comprising administering, to the subject, an effective amount of nimesulide.

2. The method of claim 1 where the subject is at risk for developing amyotrophic lateral sclerosis due to a positive family history of amyotrophic lateral sclerosis.

3. The method of claim 1 where the subject is at risk for developing amyotrophic lateral sclerosis due to the presence of a genetic mutation in the subject that has been positively correlated with amyotrophic lateral sclerosis.

4. The method of claim 3 wherein the genetic mutation is in the gene encoding superoxide dismutase.

5. The method of claim 1 where the subject is at risk for developing amyotrophic lateral sclerosis due to an environmental disclosure.

6. The method of claim 1 where the subject has a diagnosis of amyotrophic lateral sclerosis.

7. The method of claim 1 where the amount of nimesulide administered is 200 mg per day.

8. The method of claim 2 where the amount of nimesulide administered is 200 mg per day.

9. The method of claim 3 where the amount of nimesulide administered is 200 mg per day.

10. The method of claim 4 where the amount of nimesulide administered is 200 mg per day.

11. The method of claim 5 where the amount of nimesulide administered is 200 mg per day.

12. The method of claim 6 where the amount of nimesulide administered is 200 mg per day.

13. A method of treating a human subject diagnosed with amyotrophic lateral sclerosis, comprising administering, to the subject, an amount of nimesulide effective in delaying the onset of impairment of a motor function not measurably impaired prior to the initiation of of the treatment.

14. The method of claim 13 where the amount of nimesulide administered is 200mg per day.

15. An assay method for determining the effect of a test agent on the progression of motor function impairment, comprising:
(i) administering, to a mouse which serves as a murine model of motor system disease, the test agent; and
(ii) determining the level of expression, in nervous system tissue of the mouse, of a protein having a molecular weight of about 4.5-6.0 kDa and having a negative charge at pH 9, and
(iii) comparing the level determined in step (ii) with the level of the protein in a control mouse,
wherein a decrease in the level of the protein in the mouse treated with the test agent relative to the level in the control mouse has a positive correlation with the ability of the test agent to delay motor function impairment.

16. The assay method of claim 15, where the murine model is a model for amyotrophic lateral sclerosis.

17. The assay method of claim 16, where the murine model has a mutation in the superoxide dismutase gene.

18. The assay method of claim 17, where the murine model is the SOD1 mutant line.

19. The assay method of claim 15, where the murine model is a model for spinal cord injury.
